(19) United States
(12) Patent Application Publication
(10) Pub. No.: US 2024/0148662 A1
(43) Pub. Date: May 9, 2024

(54) METHODS AND SYSTEMS FOR MAKING POLYMER MICROSPHERES

(71) Applicant: Oakwood Laboratories, LLC, Oakwood Village, OH (US)

(72) Inventors: Rachel Galaska, Willoughby, OH (US); Tracy Richey, Stow, OH (US)

(21) Appl. No.: 18/548,702

(22) PCT Filed: Mar. 2, 2022
(86) PCT No.: PCT/US22/70918

§ 371 Date: Sep. 1, 2023

Related U.S. Application Data
(60) Provisional application No. 63/159,523, filed on Mar. 11, 2021.

(51) Int. Cl. A61K 9/50; A61K 31/4178; A61K 31/4422; A61K 31/519; A61K 31/5513

(52) U.S. Cl. CPC A61K 9/5089; A61K 9/5026; A61K 31/4178; A61K 31/4422; A61K 31/519; A61K 31/5513

(57) ABSTRACT

A method for making active pharmaceutical ingredient (“API”)-encapsulated polymer microspheres is provided, comprising: (A) preparing a dispersed phase (“DP”), comprising the steps of: (1) dissolving an API in a first solvent in a first vessel to form an API solution; (2) dissolving a biodegradable polymer in a second solvent in a second vessel to form a polymer solution; (3) combining the API solution and the polymer solution; and (4) mixing the combined solution to form the DP; (B) introducing the DP into a homogenization unit, wherein the time between the combining and the introducing consists of a predetermined contact time that is sufficiently short that the polymer does not undergo significant degradation attributable to the API, and the API does not undergo significant degradation or morphologic changes attributable to the polymer; (C) contacting the DP with a continuous phase (“CP”) in the homogenization unit; and (D) homogenizing the combined DP and CP.

### CROSS-REFERENCE TO RELATED APPLICATIONS

This application claims priority from U.S. Provisional Patent Application No. 63/159,523, filed on Mar. 11, 2021, which is incorporated by reference herein in its entirety.

### BACKGROUND

Polymer microspheres can be used to deliver medication in a rate-controlled and sometimes targeted manner. In its simplest form, medication is released from the polymer microsphere by an active pharmaceutical ingredient (an “API”) diffusing from the polymer, by degradation of the polymer matrix, or both.

Generally, during a continuous polymer microsphere formation process, a dispersed phase (a “DP”) and a continuous phase (a “CP”) are brought together at well-defined flow rates into a fluid shear stress zone to produce microspheres. See, e.g., U.S. Pat. No. 11,167,256 and U.S. Patent Publication No. US20220054420A1, each of which is incorporated by reference herein in its entirety. The DP typically comprises the API dispersed or dissolved in an organic solution, along with a dissolved polymer. The CP typically comprises water and, optionally, a surfactant, such as polyvinyl alcohol (“PVA”).

For some API and polymer combinations, the API has been observed to cause degradation of the polymer while in the DP—that is, prior to formation of the polymer microspheres. For example, when using risperidone as the API, it has been observed that the drug engages in a nucleophilic attack on the polymer in solution, leading to degradation of the polymer and reduced or unpredictable drug release times. One risperidone commercial product, Risperdal® Consta®, requires a high molecular weight polymer to overcome these issues.

In other API and polymer combinations, the API has been observed to undergo polymorphism of the API, e.g., shifting from one crystalline form to another or from a crystalline form to an amorphous form. For example, during the encapsulation of nimodipine drug powder, it has been observed that the non-dissolved drug crystal changes form while in the DP phase, which has a negative effect on the processability of the formulation. Such negative effects may include, for example, low encapsulation efficiencies and low batch yields.

While these issues may not be particularly acute and/or apparent at a lab scale, they become so as the scale of batches increases and the exposure time of the API to the polymer in the DP increases. A formulation that cannot be scaled up has limited value in the pharmaceutical field.

Thus, a need exists for methods to limit the exposure time between certain APIs and certain polymers in solution during formation of polymer microspheres, and more particularly during formation of the DP, to minimize scale up issues, such as polymer degradation or API degradation, including API polymorph changes.

### SUMMARY

A “DP On-Demand” method is provided for making API-encapsulated polymer microspheres. In one aspect, the method comprises: (A) preparing a DP, comprising the steps of: (1) dissolving an API in a first solvent in a first vessel to form an API solution; (2) dissolving a biodegradable polymer in a second solvent in a second vessel to form a polymer solution; (3) combining the API solution and the polymer solution; and (4) mixing the combined solution to form the DP; (B) introducing the DP into a homogenization unit, wherein the time between the combining and the introducing consists of a predetermined contact time that is sufficiently short that the biodegradable polymer does not undergo significant degradation attributable to the API, and the API does not undergo significant degradation or changes attributable to the polymer; (C) contacting the DP with a CP in the homogenization unit; and (D) homogenizing the combined DP and CP.

In another aspect, a system is provided for making API-encapsulated polymer microspheres. In one aspect, the system comprises: a first vessel, configured to contain and dispense an API solution; a second vessel, configured to contain and dispense a biodegradable polymer solution; a static mixer, configured to receive and mix the API solution and the polymer solution; a first pump, configured to pump the API solution from the first vessel to the static mixer; a second pump, configured to pump the polymer solution from the second vessel to the static mixer; a third vessel, configured to contain and dispense a CP; a homogenization unit; a third pump, configured to pump the continuous phase into the homogenization unit; a controller, configured to cause the first pump, the second pump, the static mixer, the third pump, and the homogenizer to act in concert such that a total amount of time between a pumping of the API solution and a pumping of the polymer solution into the static mixer, and a pumping of the continuous phase into the homogenizer, is less than a predetermined time.

### BRIEF DESCRIPTION OF THE FIGURES

In the accompanying figures, structures are illustrated that, together with the detailed description provided below, describe example aspects of the claimed invention. Like elements are identified with the same reference numerals. Elements shown as a single component may be replaced with multiple components, and elements shown as multiple components may be replaced with a single component. The drawings are not to scale, and the proportion of certain elements may be exaggerated for the purpose of illustration.

FIG. 1 is a schematic representation of an example system for implementing the “DP On-Demand” method for making a DP for use in a polymer microsphere formulation.

FIG. 2 is a schematic representation of an example downstream processing system for making a polymer microsphere formulation using a DP made according to the DP On-Demand method.

### DETAILED DESCRIPTION

Methods for making polymer microspheres are provided. More specifically, “DP On-Demand” methods are provided for minimizing API/polymer exposure time during formation of the polymer microspheres, thereby minimizing at least one of polymer or API degradation and API polymorph changes during formation of the polymer microspheres. In one aspect, the DP On-Demand method comprises: (A) preparing a DP, comprising the steps of: (1) dissolving an API in a first solvent in a first vessel to form an API solution; (2) dissolving a biodegradable polymer in a second solvent in a second vessel to form a polymer solution; (3) combining the API solution and the polymer solution; and (4) mixing the combined solution to form the DP; (B) introducing the DP into a homogenization unit, wherein the time between the combining and the introducing consists of a predetermined contact time that is sufficiently short that the polymer does not undergo significant degradation attributable to contact with the API and/or the API does not undergo significant degradation or polymorph changes attributable to contact with the polymer; (C) contacting the DP with a CP in the homogenization unit; and (D) homogenizing the combined DP and CP. In one aspect, a degradation of the polymer is “significant” if the polymer's molecular weight in the polymer microsphere that is at least 20% less than the raw polymer's initial molecular weight prior to contact with the API. In one aspect, a degradation of the API is “significant” if the impurity levels of the API increase by 0.10% relative to the initial API purity or the API undergoes a polymorphic change that results in a reduction in encapsulation efficiency of the API.

In one aspect, the combining comprises pumping the API solution and the polymer solution into a y-connector or equivalent structure. In one aspect, the mixing comprises mixing in a static mixer.

In one aspect, the contacting comprises pumping the CP into the homogenizing unit. In one aspect, the CP comprises water. In one aspect, the CP comprises an aqueous solution comprising a surfactant. In one aspect, the CP comprises an aqueous solution comprising PVA. In one aspect, the CP comprises between about 0.20 and about 1.5 wt % PVA solution prior to the contacting. In one aspect, the CP comprises about 0.35 wt % PVA solution prior to the contacting. In one aspect, the CP comprises about 1.0 wt % PVA solution prior to the contacting.

In one aspect, the predetermined time is less than about one minute. In another aspect, the predetermined time is less than about 30 seconds, less than about 20 seconds, or less than about 15 seconds.

### Biodegradable Polymers

Suitable biodegradable polymers may include a polylactic acid (“PLA”), a poly(lactic-co-glycolic acid) (“PLGA”), a polyesteramide, a polyanhydride, a polyacetal, a poly(ortho ester), a polyphosphoester, a polycaprolactone, a polycarbonate, and co- and tri-block polymers of any of them. In some aspects, the biodegradable polymer comprises a PLGA. In some aspects, the biodegradable polymer may comprise a co-polymer having a co-monomer ratio for lactide to glycolide content of about 50:50, about 55:45, about 75:25, about 85:15, less than 100:0, and any ratio in between.

The biodegradable polymer may have an average molecular weight of from about 10 kDa to about 300 kDa. In one aspect, the inherent viscosity (“IV”) of the polymer may be from about 0.10 to about 3.0 dL/g.

### APIs

The API may be any suitable pharmaceutical ingredient where there is an advantage to minimizing the contact time between the API and the polymer during polymer microsphere formation. Suitable APIs may include, for example, risperidone, ondansetron, nimpodipine, leuprolide acetate, octreotide acetate, and olanzapine.

### Solvents

The formation of the API solution and the polymer solution may be accomplished using various solvent systems, with solvents necessary to solubilize or suspend the API and solubilize the biodegradable polymer. For brevity, and because the methods are equally applicable to either, the phrase “API solution” contemplates either or both of a solution and a suspension. Further, the phrases “first solvent” and “second solvent” are used herein to signify the initial separateness of the API solution and the polymer solution. Unless otherwise specified, the first solvent and the second solvent may be the same (e.g., both may be dichloromethane or “DCM”) or they may be different. The first solvent and the second solvent may also each separately be a mixture of solvents. Suitable solvents may include, for example, DCM, benzyl alcohol (“BA”), chloroform, methanol, ethyl acetate, acetic acid, acetone, acetonitrile, acetyl acetone, acrolein, acrylonitrile, allyl alcohol, 1,3-butanediol, 1,4-butanediol, 1-butanol, 2-butanol, tert-butanol, 2-butoxyethanol, n-butyl amine, butyl dioxitol acetate, butyraldehyde, butyric acid, 2-chloroethanol, diacetone alcohol, diacetyl, diethylamine, diethylene glycol diethyl ether, diethylene glycol dimethyl ether, diethylene glycol monobutyl ether, diethylene glycol monobutyl ether acetate, diethylene glycol monoethyl ether, diethylene glycol monoethyl ether acetate, diethylene glycol monomethyl ether, N,N-diethylnicotinamide, dimethyl sulfoxide, N,N-dimethylacetamide, N,N-dimethylformamide, 1,4-dioxane, 2-ethoxyethanol, 2-ethoxyethyl acetate, ethyl formate, ethylene glycol methyl ether acetate, formic acid, furfural, glycofurol, hexylene glycol, isobutanol, isopropyl alcohol, 2,6-lutidine, methyl acetate, methyl ethyl ketone, methyl isopropyl ketone, methyl propionate, N-methylpyrrolidone, morpholine, tert-pentanol, 2-picoline, 3-picoline, 4-picoline, piperidine, 1-propanol, propionaldehyde, propylene oxide, pyridine, pyrimidine, pyrrolidine, tetrahydrofuran, tetramethylurea, triacetin, triethylene glycol, trimethyl phosphate, and combinations thereof.

### Homogenization Units

As used herein, the term “homogenize” is meant to include homogenization and/or emulsification. Thus, for brevity, and because the methods are equally applicable to either, the phrase “homogenization unit” contemplates a system or apparatus that can homogenize the DP and the CP, emulsify the DP and the CP, or both, which systems and apparatuses are known in the art. For example, in one aspect, the homogenization unit is of the type of system described in U.S. Pat. No. 11,167,256, which is incorporated by reference herein in its entirety. In one aspect, the homogenization unit is a membrane emulsifier or a second static mixer.

### EXAMPLES

### Example 1—General DP On-Demand Preparation of Polymer Microspheres

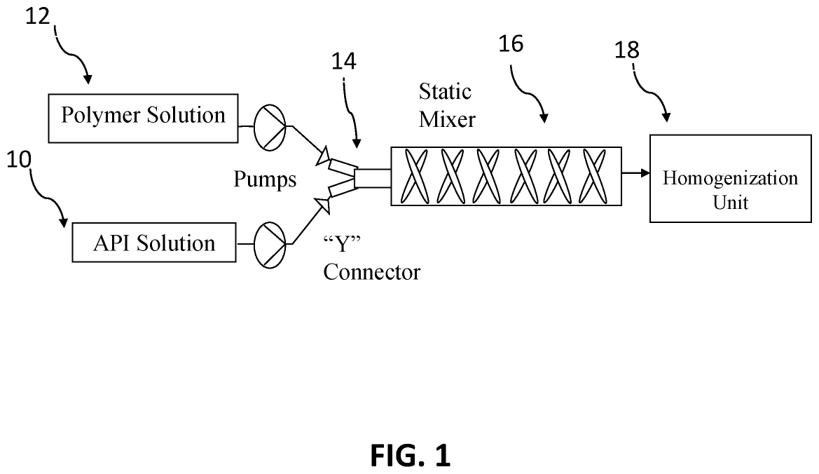

Referring to FIG. 1, an API solution 10 and a polymer solution 12 are prepared in separate vessels. For the API solution 10, an API is dissolved, dispersed, or both in a first solvent. For the polymer solution 12, a polymer is dissolved in a second solvent. The API solution 10 and the polymer solution 12 are each pumped, at defined flow rates, into a “Y” connector 14, where they are mixed in-line using a static mixer 16 to form the DP. The DP is introduced into a homogenization unit 18 (e.g., a Levitronix® BPS-i100 integrated pump system).

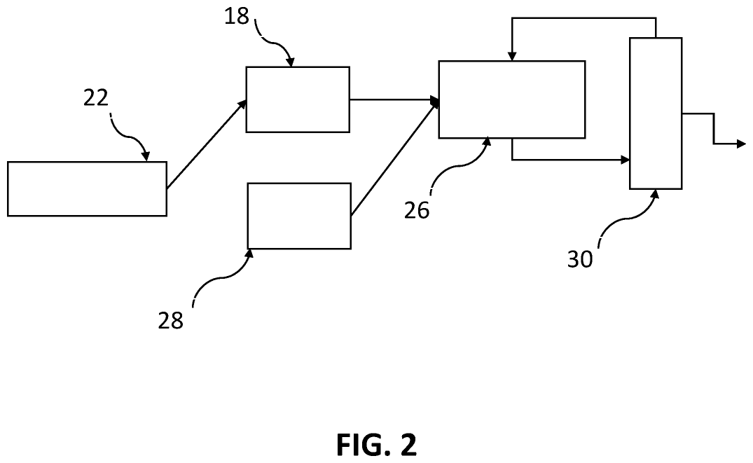

The DP may be formulated immediately (e.g., less than about 1 minute, but in any event, less than an amount of time that would permit the API to significantly degrade the polymer or permit the API to undergo an undesirable polymorph change or degradation) before it is combined with a CP. The CP may comprise an aqueous solution optionally comprising a surfactant, such as PVA. Thus, with reference to FIG. 2, the CP 22 is pumped at a defined flow rate into the homogenization unit 18 and into contact with the DP.

The formed or forming microspheres exit the homogenization unit 18 and enter a solvent removal vessel (SRV) 26. Water may be added to the SRV 26 from a water dilution composition vessel 28 to reduce the organic solvent level. See, e.g., U.S. Pat. No. 9,017,715, which is incorporated by reference herein in its entirety. The resulting suspension is mixed in the SRV 26. After the DP in the homogenization unit 18 has been exhausted, the CP 22 and water dilution composition vessel 28 pumps are stopped, and washing steps are initiated. In some aspects, solvent removal is achieved using water washing and a hollow fiber filter 30.

The washing steps may comprise washing the microsphere suspension with room temperature water, followed by washing the suspension with hot water (about 35-39° C.) with several volume exchanges before cooling the suspension back down to room temperature.

The washed microspheres are collected and lyophilized overnight (Virtis) to remove moisture. The resulting API-encapsulated polymer microspheres are a free-flowing off-white bulk powder.

### Example 2—Ondansetron Encapsulation in Polymer Microspheres Using the DP On-Demand Method

One batch of ondansetron-encapsulated microspheres was prepared according to the DP On-Demand method described in Example 1 (Batch #1), using Resomer® RG 503 H, Poly(D,L-lactide-co-glycolide), 50:50, acid end-capped, as the polymer, and BA/DCM (1:1) as the solvent system. The API solution flow rate was 28 mL/min, and the polymer solution flow rate was 22 mL/min. A control batch (Batch #2) was made using a DP that was prepared by mixing the polymer, API, and both solvents in one vessel for approximately four hours before it was combined with the CP, to approximate the conditions (i.e. exposure times) of a large scale batch. The DP solution flow rate was 50 mL/min. The CP flow rate was 4 L/min for a CP:DP ratio for both batches of 80:1. The characterization data for Batch #s 1 and 2 are shown in Table 1:

TABLE 1

Batch #

| | |
|---|---|
| 1 | |
| DP On- | 2 |
| Demand | Control |

| | | |
|---|---|---|
| Formulations | Polymer | 503H |
| | Monomer Ratio | 50:50 |
| | Polymer IV (dL/g) | 0.34 |
| | Target Drug Load (%) | 20.0 |

| | | | |
|---|---|---|---|
| Analytical | Drug Load (%) | 22.0 | 18.0 |
| | Encapsulation | 110 | 90 |
| | Efficiency (%) | | |

| | |
|---|---|
| Impurities (%) | N/A |

| Residual Solvent | | 2.5/ND | 1.5/ND |
|---|---|---|---|
| BA/DCM (%) | | | |
| Particle | Dᵥ10 | 11 | 10 |
| Size | Dᵥ50 | 23 | 21 |
| (μm) | Dᵥ90 | 39 | 41 |

| | | |
|---|---|---|
| Sample MW (kDa) | 29.7 | 12.0 |

| | |
|---|---|
| Polymer MW (kDa) | 30.5 |

As can be seen from Table 1, Batch #1 has a ˜2.5× higher molecular weight than the Batch #2 control, indicating that the DP On-Demand method is surprisingly effective to reduce the degradation of the polymer during microsphere formation.

### Example 3—Ondansetron Encapsulation in Polymer Microspheres Using the DP on Demand Method

One batch of ondansetron-encapsulated microspheres was prepared according to the DP On-Demand method described in Example 1 (Batch #3), using Resomer® RG 753 H, Poly(D,L-lactide-co-glycolide), 75:25, acid end-capped, as the polymer, and BA/DCM (1:1) as the solvent system. The API solution flow rate was 28 mL/min, and the polymer solution flow rate was 22 mL/min. A control batch (Batch #4) was made using a DP that was prepared by mixing the polymer, API, and both solvents in one vessel for approximately four hours before it was combined with the CP, to approximate the conditions of a large scale batch. The DP solution flow rate was 50 mL/min. The CP flow rate was 4 L/min for a CP:DP ratio for both batches of 80:1. The characterization data for Batch #s 3 and 4 are shown in Table 2:

TABLE 2

Lot

| | |
|---|---|
| 3 | 4 |
| DP-OD | Control |

| | | |
|---|---|---|
| Formulations | Polymer | Evonik 753H |
| | Monomer Ratio | 75:25 |
| | Polymer IV (dL/g) | 0.39 |
| | Target Drug Load (%) | 20 |

| | | | |
|---|---|---|---|
| Analytical | Drug Load (%) | 18.0 | 16.4 |
| | Encapsulation | 90 | 82 |
| | Efficiency (%) | | |
| | Residual Solvent | 1.9/ND | 2.6/ND |
| | BA/DCM (%) | | |

| | | | |
|---|---|---|---|
| Particle | Dᵥ10 | 11 | 10 |
| Size | Dᵥ50 | 27 | 23 |
| (μm) | Dᵥ90 | 54 | 41 |

| | | |
|---|---|---|
| Sample MW (kDa) | 36 | 23 |

| | |
|---|---|
| Polymer MW (kDa) | 38 |

As can be seen from Table 2, Batch #3 has a ˜1.5× higher molecular weight than the Batch #4 control, indicating that the DP On-Demand method is surprisingly effective to reduce the degradation of the polymer during microsphere formation.

### Example 4—Ondansetron Encapsulation in Polymer Microspheres Using the DP on Demand Method

One batch of ondansetron-encapsulated microspheres was prepared according to the DP On-Demand method described in Example 1 (Batch #5), using Viatel™ DLG 5505A, Poly(D,L-lactide-co-glycolide), 55:45, acid end-capped, as the polymer, and BA/DCM (1:1.25) as the solvent system. The API solution flow rate was 27 mL/min, and the polymer solution flow rate was 23 mL/min. A control batch (Batch #6) was made using a DP that was prepared by mixing the polymer, API, and both solvents in one vessel for approximately four hours before it was combined with the CP, to approximate the conditions of a large scale batch. The DP solution flow rate was 50 mL/min. The CP flow rate was 4 L/min for a CP:DP ratio for both batches of 80:1. The characterization data for Batch #s 5 and 6 are shown in Table 3:

TABLE 3

Lot

| | |
|---|---|
| 5 | 6 |
| DP-OD | Control |

| | | |
|---|---|---|
| Formulations | Polymer | Viatel ™ DLG 5505A |
| | Monomer Ratio | 55:45 |
| | Polymer IV (dL/g) | 0.59 |
| | Target Drug Load (%) | 20 |

| | | | |
|---|---|---|---|
| Analytical | Drug Load (%) | 15.4 | 17.2 |
| | Encapsulation | 77 | 86 |
| | Efficiency (%) | | |
| | Residual Solvent | 1.9/ND | 2.6/ND |
| | BA/DCM (%) | | |
| | Sample MW (kDa) | 68.1 | 24.3 |

| | |
|---|---|
| Polymer MW (kDa) | 76.1 |

As can be seen from Table 3, Batch #5 has a ˜2.8× higher molecular weight than the Batch #6 control, indicating that the DP On-Demand method is surprisingly effective to reduce the degradation of the polymer during microsphere formation.

The aspects disclosed herein are not intended to be exhaustive or to be limiting. A skilled artisan would acknowledge that other aspects or modifications to instant aspects can be made without departing from the spirit or scope of the invention. The aspects of the present disclosure, as generally described herein and illustrated in the figures, can be arranged, substituted, combined, separated, and designed in a wide variety of different configurations, all of which are contemplated herein.

Unless otherwise specified, “a,” “an,” “the,” “one or more of,” and “at least one” are used interchangeably. The singular forms “a”, “an,” and “the” are inclusive of their plural forms. The recitations of numerical ranges by endpoints include all numbers subsumed within that range (e.g., 1 to 5 includes 1, 1.5, 2, 2.75, 3, 3.80, 4, 5, etc.). The terms “comprising” and “including” are intended to be equivalent and open-ended. The phrase “consisting essentially of” means that the composition or method may include additional ingredients and/or steps, but only if the additional ingredients and/or steps do not materially alter the basic and novel characteristics of the claimed composition or method. The phrase “selected from the group consisting of” is meant to include mixtures of the listed group.

When reference is made to the term “each,” it is not meant to mean “each and every, without exception.” For example, if reference is made to microsphere formulation comprising polymer microspheres, and “each polymer microsphere” is said to have a particular API content, if there are 10 polymer microspheres, and two or more of the polymer microspheres have the particular API content, then that subset of two or more polymer microspheres is intended to meet the limitation.

The term “about” in conjunction with a number is intended to include ±10% of the number. This is true whether “about” is modifying a stand-alone number or modifying a number at either or both ends of a range of numbers. In other words, “about 10” means from 9 to 11. Likewise, “about 10 to about 20” contemplates 9 to 22 and 11 to 18. In the absence of the term “about,” the exact number is intended. In other words, “10” means 10.

The phrase “operatively connected” is “a general descriptive claim term frequently used in patent drafting to reflect a functional relationship between claimed components,” that is, the term “means the claimed components must be connected in a way to perform a designated function.” MPEP 2173.05(g).

What is claimed is:

1. A method for preparing an active pharmaceutical ingredient (“API”)-encapsulating polymer microsphere formulation, the method comprising:
(A) preparing a dispersed phase, comprising the steps of:
(1) dissolving and/or suspending an API in a first solvent in a first vessel to form an API solution;
(2) dissolving a biodegradable polymer in a second solvent in a second vessel different from the first vessel to form a polymer solution;
(3) combining the API solution and the polymer solution; and
(4) mixing the combined solution to form the dispersed phase;
(B) introducing the dispersed phase into a homogenization unit, wherein the time between the combining and the introducing consists of a predetermined contact time that is sufficiently short that the polymer does not undergo significant degradation attributable to contact with the API, and/or the API does not undergo significant degradation or morphologic changes attributable to contact with the polymer;
(C) contacting the dispersed phase with a continuous phase in the homogenization unit; and
(D) homogenizing the combined dispersed phase and continuous phase.

2. The method according to claim 1, wherein the combining comprises pumping the API solution and the polymer solution into a y-connector.

3. The method according to claim 1, wherein the mixing comprises mixing in a static mixer.

4. The method according to claim 1, wherein the contacting comprises pumping the continuous phase into the homogenizing unit.

5. The method according to claim 1, wherein the continuous phase comprises water.

6. The method according to claim 1, wherein the continuous phase comprises an aqueous solution comprising polyvinyl alcohol in a concentration of from about 0.35 wt % to about 1.0 wt % prior to the contacting.

7. The method according to claim 1, wherein the predetermined contact time is less than about one minute.

8. The method according to claim 1, wherein the predetermined contact time is less than about 30 seconds.

9. The method according to claim 1, wherein the predetermined contact time is less than about 15 seconds.

10. A system for preparing an active pharmaceutical ingredient (“API”)-encapsulating polymer microsphere formulation, the system comprising:
a first vessel containing an API solution;
a second vessel different from the first vessel containing a polymer solution;
a static mixer;
a first pump, operatively connected to the first vessel and the static mixer;
a second pump, operatively connected to the second vessel and the static mixer;
a third vessel different from the first vessel and the second vessel containing a continuous phase;
a homogenization unit;
a third pump, operatively connected to the third vessel and the homogenization unit; and
a controller, configured to cause the first pump, the second pump, the static mixer, the homogenizer, and the third pump to act in concert such that a total amount of time between a pumping of the API solution and a pumping of the polymer solution into the static mixer, and a pumping of the continuous phase into the homogenizer, is less than a predetermined time.

11. An active pharmaceutical ingredient (“API”)-encapsulated polymer microsphere formulation, prepared by a process comprising:
(A) preparing a dispersed phase, comprising the steps of:
(1) dissolving and/or suspending an API in a first solvent in a first vessel to form an API solution;
(2) dissolving a biodegradable polymer in a second solvent in a second vessel different from the first vessel to form a polymer solution;
(3) combining the API solution and the polymer solution; and
(4) mixing the combined solution to form the dispersed phase;
(B) introducing the dispersed phase into a homogenization unit, wherein the time between the combining and the introducing consists of a predetermined contact time that is sufficiently short that the polymer does not undergo significant degradation attributable to contact with the API, and/or the API does not undergo significant degradation or morphologic changes attributable to contact the polymer;
(C) contacting the dispersed phase with a continuous phase in the homogenization unit; and
(D) homogenizing the combined dispersed phase and continuous phase.

12. The formulation according to claim 11, wherein the API is ondansetron.

13. The formulation according to claim 11, wherein the API is risperidone.

14. The formulation according to claim 11, wherein the API is nimodipine.

15. The formulation according to claim 11, wherein the API is olanzapine.
